(19) United States
(12) Patent Application Publication
(10) Pub. No.: US 2025/0327032 A1
(43) Pub. Date: Oct. 23, 2025

(54) AGENT FOR AVOIDING IMMUNE RESPONSE OF TRANSGENIC CELLS

(71) Applicant: HYPERION DRUG DISCOVERY CO., LTD., Nara (JP)

(72) Inventors: Kenichi TAMURA, Nara (JP); Rina AKAHIRA; Kazuhiro TAKEKITA, Nara (JP)

(21) Appl. No.: 18/724,823

(22) PCT Filed: Dec. 27, 2022
(86) PCT No.: PCT/JP2022/048331

§ 371 Date: Dec. 3, 2024

(30) Foreign Application Priority Data
Dec. 28, 2021 (JP) 2021-215047
Jun. 29, 2022 (JP) 2022-104279

(51) Int. Cl. C12N 5/0775; C12N 5/077; C12N 15/113

(52) U.S. Cl. CPC C12N 5/0662; C12N 5/0658; C12N 15/1136; C12N 2310/14; C12N 2500/38; C12N 2501/60; C12N 2510/00

(57) ABSTRACT

The present invention addresses the problem of avoiding an immune response caused by gene transfection. The present inventors have found, after intensive studies to solve the above problem, that it is possible to avoid apoptosis due to innate immunity and increase the number of transgenic cells by suppressing innate immunity in cells into which a gene is transfected. On the basis of this finding, further studies have been conducted to complete the present invention.

### TECHNICAL FIELD

The present invention relates to the introduction of genes into cells.

### BACKGROUND ART

Introduction of genes is a technology for introducing desired genes into cells, and forms the basis of genetic engineering. Such technology of introduction of genes has been applied to functional analysis of genes, production of transgenic crops, gene therapy, etc. However, it is known that when introducing genes into cells or tissues, cell death occurs due to innate immunity caused by the transgene itself (Non-Patent Literature 1).

In order to avoid such cell death by innate immunity, at the time of transfection into cells, various modified nucleosides such as N1-methylpseudouridine and 5-methylcytidine have been used for the messenger RNA (hereinafter referred to as mRNA) to be introduced (Non-Patent Literature 2). Here, during translation, the introduced mRNA terminates protein biosynthesis using a stop codon that means the termination of translation process. However, when the uridine in the stop codon (UAA, UAG, or UGA) is pseudouridylated, it is known that the ribosome reads through the modified stop codon (Non-Patent Literature 3). In addition, self-amplifying mRNA technology, which amplifies administered mRNA in vivo in the body, is attracting attention as a next-generation mRNA pharmaceutical; however, since it can weaken RDRP-mediated mRNA amplification in target cells and the effect of modified nucleosides is lost in the first round of amplification, it has been stated that the use of modified nucleosides is not an option for the self-amplifying RNA (Non-Patent Literature 4).

### CITATION LIST

### Non-Patent Literature

[Non-Patent Literature 1] Katalin Kariko et al., Mol. Ther. 2008 Nov.; 16 (11): 1833-1840
[Non-Patent Literature 2] Oliwia Andries et al., Journal of Controlled Release 217 (2015) 337-344
[Non-Patent Literature 3] Hironori Adachi, et al., RNA (2020) 26: 1247-1256
[Non-Patent Literature 4] Giulietta Maruggi, et al., Molecular Therapy (2019) 27: 757-772

### SUMMARY OF INVENTION

### Problems to be Solved by Invention

The present invention addresses the problem of avoiding an immune response due to introduction of genes.

### Means of Solving Problems

The present inventors have focused on the fact that cell death by innate immunity caused by the transgene itself makes it difficult to obtain a sufficient amount of cells into which target genes have been introduced, and in the course of intensive research to solve the above problem, found that cell death by innate immunity can be avoided and the yield of transgenic cells can be increased by suppressing innate immunity in the cells into which genes are introduced; on the basis of this finding, the present inventors have conducted further research and completed the present invention.

That is, the present invention relates to the following.
[1] An agent for avoiding an immune response due to introduction of genes, by means of suppressing the innate immunity against the transgene of cells into which the gene is introduced.
[2] The agent according to [1], wherein the innate immunity is suppressed by inhibiting NFκB or inhibiting the expression of a gene related to innate immunity.
[3] The agent according to [2], wherein NFκB is suppressed by adding an NFκB nuclear translocation inhibitor.
[4] The agent according to [3], wherein the NFκB nuclear translocation inhibitor is JSH-21, JSH-23, vitamin E, calcitriol, calcifediol, vitamin D, vitamin C, vitamin B6, rolipram, SN50, or a derivative thereof.
[5] The agent according to [2], wherein the gene related to innate immunity is TNF, IFN, p50, p65, IRF3, IL-6, IL-12, TLR, MyD88, or TRIF.
[6] The agent according to [5], wherein the expression of a gene related to innate immunity is inhibited using siRNA.
[7] The agent according to any one of [1] to [6], wherein the cells are human cells.
[8] The agent according to [7], wherein the human cells are human myoblasts, human mesenchymal stem cells, or human T cells.
[9] The agent according to any one of [1] to [6], wherein the transgene is single-stranded or double-stranded.
[10] Transgenic cells obtained using the agent according to any one of [1] to [6].
[11] An agent for avoiding an immune response due to introduction of genes, by means of administering a transgene and an NFκB inhibitor or an expression inhibitor of a gene related to innate immunity.
[12]A method for avoiding an immune response due to introduction of genes, comprising suppressing the innate immunity against the transgene of cells into which the gene is introduced in vitro.

### Advantageous Effects of Invention

By using the agent of the present invention, immune response due to transgenic cells can be more easily avoided than using modified nucleosides such as pseudouridine, thereby increasing the yield of transgenic cells. In addition, by using the agent of the present invention, the yield can be easily increased, so that the time and cost required to produce a sufficient amount of cells into which a gene of interest is introduced can be reduced. Furthermore, by using the agent of the present invention, the allowable amount of the transgene in the cell is improved, so that the efficiency of introduction can be further improved.

By using the agent of the present invention, the yield of transgenic cells can be increased without using modified nucleosides such as pseudouridine; therefore, an effort to prevent read-through of stop codon, which has been required when using pseudouridine as a modified nucleoside, can be omitted. In addition, by using the agent of the present invention, it is expected that cell death caused by innate immunity can be avoided even in cases where the use of modified nucleosides is difficult such as in self-amplifying mRNA technology. Furthermore, by using both the agent of the present invention and a modified nucleoside such as pseudouridine, the innate immunity of cells can be more strongly suppressed.

In the cells obtained using the agent of the present invention, genes have already been introduced with a high probability; and for example, such transgenic cells of the present invention can be administered to subjects who require cells that have been differentiated, transdifferentiated, reprogrammed, etc. from somatic cells or somatic stem cells, or to subjects who require cells endowed with arbitrary gene expression ability. Alternatively, by using the above cells in combination with introduction of genes into living body, it can be expected that the efficiency of introduction into living body will be improved and adverse events will be avoided.

### BRIEF DESCRIPTION OF DRAWINGS

FIG. 1 is a schematic diagram of a TERT plasmid introduced into human mesenchymal stem cells using the agent of the present invention. In the figure, EF1A represents the EF1a promoter, hTERT represents the human TERT gene (SEQ ID NO: 3), SV40 late pA represents the simian virus 40 late polyA addition signal, pUC on represents the pUC replication origin, Ampicillin represents the sequence of ampicillin resistance gene, respectively.

FIG. 2 is a graph showing the number of GFP-positive cells when GFP mRNA containing uridine was introduced into human mesenchymal stem cells using a control or a culture solution supplemented with JSH-23 (0.1, 1, or 10 μM). The vertical axis shows the respective relative number of cells when the number of cells in the control is set to 1.

FIG. 3 is a graph showing the number of GFP-positive cells when GFP mRNA containing pseudouridine instead of uridine was introduced into human mesenchymal stem cells using a control or a culture solution supplemented with JSH-23. The vertical axis shows the relative number of cells when the number of cells in the control is set to 1.

FIG. 4 is a graph showing the number of GFP-positive cells when GFP mRNA containing uridine was introduced into human myoblasts using a control or a culture solution supplemented with JSH-23. The vertical axis shows the respective relative number of cells when the number of cells in the control is set to 1.

FIG. 5 is a graph showing the number of GFP-positive cells when GFP mRNA containing pseudouridine instead of uridine was introduced into human myoblasts using a control or a culture solution supplemented with JSH-23. The vertical axis shows the respective relative number of cells when the number of cells in the control is set to 1.

FIG. 6 is a graph showing the number of GFP-positive cells when GFP mRNA containing uridine was introduced into human mesenchymal stem cells using a control or a culture solution supplemented with calcitriol. The vertical axis shows the relative number of cells when the number of cells in the control is set to 1.

FIG. 7 is a graph showing the number of GFP-positive cells when GFP mRNA containing pseudouridine instead of uridine was introduced into human mesenchymal stem cells using a control or a culture solution supplemented with calcitriol. The vertical axis shows the relative number of cells when the number of cells in the control is set to 1.

FIG. 8 is a graph showing the number of GFP-positive cells when GFP mRNA containing uridine was introduced into human myoblasts using a control or a culture solution supplemented with calcitriol. The vertical axis shows the relative number of cells when the number of cells in the control is set to 1.

FIG. 9 is a graph showing the number of GFP-positive cells when GFP mRNA containing pseudouridine instead of uridine was introduced into human myoblasts using a control or a culture solution supplemented with calcitriol. The vertical axis shows the relative number of cells when the number of cells in the control is set to 1.

FIG. 10 is a graph showing the number of viable cells when TERT mRNA containing uridine was introduced into human mesenchymal stem cells using a control or culture solution supplemented with JSH-23. The vertical axis shows the relative number of cells when the number of cells in the control is set to 1.

FIG. 11 is a graph showing the number of viable cells when TERT mRNA containing pseudouridine instead of uridine was introduced into human mesenchymal stem cells using a control or a culture solution supplemented with JSH-23. The vertical axis shows the relative number of cells when the number of cells in the control is set to 1.

FIG. 12 is a graph showing the number of viable cells when TERT mRNA containing uridine was introduced into human myoblasts using a control or a culture solution supplemented with JSH-23. The vertical axis shows the relative number of cells when the number of cells in the control is set to 1.

FIG. 13 is a graph showing the number of viable cells when TERT mRNA containing pseudouridine instead of uridine was introduced into human myoblasts using a control or a culture solution supplemented with JSH-23. The vertical axis shows the relative number of cells when the number of cells in the control is set to 1.

FIG. 14 is a graph showing the expression level of TNF-α in cells into which mRNA using pseudouridine was introduced and in cells into which mRNA using pseudouridine was introduced using JSH-23. The vertical axis shows the respective relative expression level when the expression level of TNF-α in the control is set to 1.

FIG. 15 is a graph showing the number of viable cells when TERT plasmid was introduced into human mesenchymal stem cells using a control or a culture solution supplemented with JSH-23. The vertical axis shows the relative number of cells when the number of cells in the control is set to 1.

FIG. 16 is a graph showing the number of GFP-positive cells when GFP viral vector was introduced into human mesenchymal stem cells using a control or a culture solution supplemented with JSH-23. The vertical axis shows the relative number of cells when the number of cells in the control is set to 1.

FIG. 17 is a graph showing the number of GFP-positive cells when GFP viral vector was introduced into human myoblasts using a control or a culture solution supplemented with JSH-23. The vertical axis shows the relative number of cells when the number of cells in the control is set to 1.

FIG. 18 is a graph showing the number of GFP-positive cells, when GFP viral vector was introduced into human myoblasts using each mixed solution (a combination of GFP viral vector: MOI 0.1, 1.5 or 10; polybrene: 0, 5 or 10; and presence or absence of calcitriol). The vertical axis shows the relative number of cells in each case when the number of cells in a mixed solution containing polybrene 5 μg/mL, viral vector with MOI of 0.1, and no calcitriol is set to 1.

FIG. 19 is a graph showing the number of GFP-positive cells, when GFP mRNA was introduced into human mesenchymal stem cells under the following three conditions: GFP mRNA: 20 pg/cell and a culture solution supplemented with JSH-23, GFP mRNA: 20 pg/cell and a culture solution without JSH-23, GFP mRNA: 10 pg/cell and a culture solution without JSH-23 (control). The vertical axis shows the relative number of cells when the number of cells in the control is set to 1.

FIG. 20 is a graph showing the number of GFP-positive cells when GFP mRNA was introduced into human mesenchymal stem cells using a lipofection solution containing each siRNA. The vertical axis shows the relative number of cells when the number of cells in the control is set to 1.

FIG. 21 is a schematic diagram of a GFP-tagged CAR-T plasmid introduced into human leukemia T cells using the agent of the invention. In the figure, CMV (CMV Promoter) represents the sequence of the human cytomegalovirus immediate enhancer/promoter, Kozak represents the Kozac translation initiation sequence, CD8-leader represents the sequence of the leader signal peptide of the T cell surface glycoprotein CD8α chain, CD19-scFV represents the single chain variable fragment sequence (SEQ ID NO: 10) derived from CD19 antibody, CD28-hinge represents the sequence of the hinge region of T cell surface glycoprotein CD28 (SEQ ID NO: 11), CD28-TM represents the sequence of the CD28 transmembrane region (SEQ ID NO: 12), CD28 represents the sequence of the CD28 intracellular costimulatory domain (SEQ ID NO: 13), CD3zeta represents the sequence of the intracellular domain of the T cell receptor CD3ζ chain, SV40 late pA represents the simian virus 40 late polyA addition signal, EGFP represents the EGFP gene (SEQ ID NO: 14), BGH pA represents the polyadenylation signal of bovine growth hormone, pUC on represents the pUC replication origin, and Ampicillin represents the sequence of the ampicillin resistance gene.

FIG. 22 is a graph showing the number of GFP-positive cells when GFP-tagged CAR-T plasmid gene was introduced into human leukemia T cells using each culture solution. The vertical axis shows the relative number of cells when the number of cells in the control is set to 1.

FIG. 23 is a graph showing the expression level of TNF-α in cells into which GFP-tagged CAR-T plasmid was introduced, and in cells into which CAR-T plasmid was introduced using JSH-23. The vertical axis shows the respective relative expression level when the expression level of TNF-α in the control is set to 1.

FIG. 24 is a graph showing the expression level of IFN-α in cells into which GFP-tagged CAR-T plasmid was introduced, and in cells into which CAR-T plasmid was introduced using JSH-23. The vertical axis shows the respective relative expression level when the expression level of IFN-α in the control is set to 1.

FIG. 25 is a graph showing the number of GFP positive cells when GFP mRNA was introduced into human mesenchymal stem cells using a control or a culture solution containing JSH-23, BAY11, dexamethasone, or 6-amino-4-(4-phenoxyphenethylamino)quinazoline. The vertical axis shows the respective relative number of cells when the number of cells in the control is set to 1.

FIG. 26 is a graph showing the number of GFP-positive cells when GFP mRNA was introduced into human mesenchymal stem cells using a control or a culture solution containing vitamin B6, vitamin C, vitamin E, or rolipram. The vertical axis shows the respective relative number of cells when the number of cells in the control is set to 1.

### EMBODIMENTS FOR CARRYING OUT THE INVENTION

Hereinafter, the present invention will be explained in detail.

The present invention relates to an agent and a method for avoiding immune response due to introduction of genes, by means of suppressing the innate immunity against transgene of cells into which the gene is introduced.

In the present invention, to suppress innate immunity, any known method for suppressing innate immune function of cells into which genes may be introduced can be used. Such methods include, but are not limited to, inhibition of factors involved in innate immune response and cell death, specifically, inhibition of NFκB, inhibition of expression of genes related to innate immunity and the like. Suppressing innate immunity may be performed by simultaneously or separately administering a transgene and a factor that suppresses innate immune function to cells, and specifically, it may be performed at the time of introducing the gene into the cells, or it may be performed at the time of culturing the cells before introducing the gene.

Inhibition of NFκB is performed, for example, by using an NFκB inhibitor. The NFκB inhibitor is, as a component of the agent of the present invention, for example, added to a culture solution of cells into which a gene can be introduced. NFκB inhibitors are generally classified into as follows according to the NFκB inhibitory effect of the compound (small molecule compound, protein, antibody, etc.) contained in the agent: NFκB nuclear translocation inhibitors, DNA binding inhibitors, IKK inhibitors, IKβ degradation agents, p65 acetylation inhibitors, NFκB transcriptional activity inhibitors, etc. As the NFκB inhibitor used in the present invention, an NFκB nuclear translocation inhibitor is preferable from the viewpoint of ensuring a higher survival rate. In the present invention, an NFκB nuclear translocation inhibitor refers to an agent comprising a compound that inhibits the action of NFκB mainly by inhibiting nuclear translocation of NFκB. For example, the NFκB nuclear translocation inhibitor may not only inhibit NFκB nuclear translocation but also have other NFκB inhibitory effects. Without being bound by a particular theory, inhibition of NFκB by an NFκB nuclear translocation inhibitor reduces the occurrence of apoptosis in cells to which the agent is administered, compared to inhibition of NFκB by an inhibitory effect other than inhibition of nuclear translocation.

In one embodiment, an NFκB nuclear translocation inhibitor is used as the NFκB inhibitor. In such embodiments, the NFκB nuclear translocation inhibitors used include those listed in Table 1 of Subash C Gupta et al., Biochim Biophys Acta. 2010; 1799(10-12): 775-787, such as JSH-21, JSH-23, vitamin E, calcitriol, calcifediol, vitamin D, vitamin C, vitamin B6, rolipram, SN50 and derivatives thereof, etc.; from the viewpoint of easy availability, JSH-23 or calcitriol is preferred. When an NFκB nuclear translocation inhibitor is added to the culture solution, its concentration may be 0.001 to 300 μM, preferably 0.1 to 10 μM. When JSH-23 is added to the culture solution as an NFκB nuclear translocation inhibitor, its concentration may be 0.001 to 300 μM, preferably 0.1 to 50 μM. When calcitriol is added to the culture solution as an NFκB nuclear translocation inhibitor, its concentration may be 0.001 to 10 μM, preferably 1 μM.

Inhibition of expression of genes related to innate immunity is performed using any known method, for example, using interfering nucleic acids, ribozymes, antisense nucleic acids, microRNAs, and single-stranded hairpin RNAs for genes related to innate immunity, and vectors expressing these; it is preferably performed by gene silencing using RNA interference. For RNA interference, siRNA or miRNA or the like is used; but in the present invention, it is preferable to use siRNA from the viewpoint of structural stability due to its double-stranded structure and ease of synthesis due to its short chain length. In the present invention, RNA interference can be performed, for example, by adding siRNA or the like as a component of the agent of the present invention to the culture solution of cells into which a gene is introduced. The introduction of siRNA can be performed according to the manufacturer's instructions. When siRNA is added to the culture solution, its concentration may be 0.1 to 100 nM, preferably 5 to 20 nM.

In addition, in the present invention, the gene targeted for expression inhibition is not particularly limited as long as it is a gene related to innate immunity in the cell. Examples of such genes include TNF, IFN, p50, p65, IRF3, IL-6, IL-12, TLR, MyD88, TRIF, etc.; from the viewpoint of specifically inhibiting the expression of inflammatory cytokines that induce cell death, TNF or IFN is preferable, and TNF-α or IFN-α is more preferable. Most preferably, the genes targeted for inhibition of expression are TNF-α and IFN-α.

In one embodiment, avoiding the immune response of introduction of genes results in an increased yield of transgenic cells in vitro. An increase in the yield of transgenic cells includes an increase in the number of transgenic cells and an increase in the efficiency of introduction of genes. Furthermore, in the present invention, increasing the yield of transgenic cells includes obtaining transgenic cells with increased yield.

The agent of the present invention can be used in the art to introduce any gene that can be normally used for introduction of genes into cells. In the present invention, the gene introduced into cells, that is, the transgene, may be single-stranded or double-stranded. Examples of the single-stranded transgene include mRNA and miRNA. Examples of the double-stranded transgene include viral vectors, plasmid DNA, and siRNA. In the present invention, the transgene is not particularly limited. For example, examples of the transgene include: genes involved in the cell cycle (CDK family, Cyclin family, p16, p21, p27, E2F and their variants, etc.), genes related to the structure of chromosomes (telomerase, TERT, ZSCAN, SV40 Large T antigen, HPV E6/E7, Ras, Rb, recombinase, integrase, nuclease, helicase, ligase, replicase, B-cell lymphoma 2, CRISPR Cas and their variants, etc.), reprogramming-related genes (Oct3/4, c-Myc, Klf4, Sox2, NANOG, ASCL1, PITX3, NURR1, LMX1A and their variants, etc.), skeletal muscle-related genes (CD56, Pax3, Pax7, Myogenin, Myf5, MyoD, Myomaker, Myomixer, Myosin Heavy Chain, Desmin, Dystrophin, Myotilin, Laminin A/C, CAV, CAPN, SGCG, TRIM32, TCAP, FKRP, EMD, PABP, DMPK, ZNF9, FCMD, POMENTI, Collagen, SEPN1, RYRI, MTM, TNNT, NEB, TPM, ACTN, GNE, DYSF, CRY AB, ACTA and their variants, etc.), genes related to growth factors (VEGF, IGF, FGF, HGF, EGF, TGF, NGF, BDNF, GDNF, BMP, PDGF, EPO, TPO, G-CSF, GM-CSF and their families and their variants, etc.), genes related to transcription factors (Runt domain including RUNX and its variants, helix-turn-helix, helix-loop-helix, zinc finger, leucine zipper, β-sheet motif and their variants, etc.), genes related to enzymes (glucose-6-phosphatase, t-PA, collagenase, alglucosidase, urate oxidase, alkaline phosphatase, glycosaminoglycan degrading enzyme, β-glucuronidase, glutamic acid carboxypeptidase, sphingomyelin phosphodiesterase, α-L-iduronidase, iduronic acid sulfatase, N-acetylgalactosamine-6-sulfatase, N-acetylgalactosamine-4-sulfatase, α-galactosidase, α-glucosidase, β-glucocerebrosidase, lysosomal acid lipase, and their variants, etc.), genes related to membrane proteins (monotopic including neprilysin, as well as polytopic and their variants, etc.), genes related to chimeric antigen receptors (CAR and its variants, etc.), antibody genes (mouse antibodies, chimeric antibodies, humanized antibodies, human antibodies, antibodies without substem indicating origin, etc.), blood coagulation-related factors, serum proteins, hormones, vaccines, interferons, erythropoietins, cytokines, toxins, fusion proteins, etc. In the present invention, for example, by introducing a gene as described above, the lifespan of the cell into which the gene has been introduced can be prolonged, or differentiation into desired cells can be promoted.

In the present invention, cells into which the gene can be introduced are not particularly limited as long as they are cells that can be normally used for introduction of genes in the art. Examples of such cells include stem cells (e.g., pluripotent stem cells, pluripotent stem cell-derived differentiated cells, mesenchymal stem cells, myoblasts, hematopoietic stem cells, etc.), somatic cells (e.g., muscle cells, blood cells (T cells, B cells, etc.), fibroblasts, nervous system cells, epidermal cells, epithelial cells, endothelial cells, osteocytes, chondrocytes, adipocytes, etc.). In the present invention, cells into which the gene can be introduced may be cells derived from any organism that can be normally used for introduction of genes in the art. Examples of such cells include cells derived from mammals. Preferably, in the present invention, cells into which the gene can be introduced are human-derived cells. More preferably, cells into which the gene can be introduced in the present invention are human mesenchymal stem cells or human cells differentiated from human mesenchymal stem cells such as human myoblasts, human T cells, or human muscle cells.

In the present invention, the method for introducing a gene into cells can be performed using any known method. Examples of such methods include lipofection method, electroporation method, polybrene method and the like.

In the present invention, when the lipofection method is used, cells into which the gene is introduced are seeded at about 1,000 to 100,000 cells/cm². Preferably, from the viewpoint of stabilizing the final yield, the cells into which the gene is introduced are seeded at about 5,000 to 20,000 cells/cm². When the cells into which the gene is introduced are human mesenchymal stem cells or human T cells, more preferably, the cells are seeded at 1×10⁴ cells/cm², and when the cells are human myoblasts, more preferably they are seeded at 2×10⁴ cells/cm².

For example, when mRNA is introduced into cells using the lipofection method, the mRNA is mixed with a lipofection reagent such as Lipofectamine (Thermo Fisher Scientific) at an amount of 1 to 100 pg/cell, and then added to the cells into which the gene is introduced. Preferably, from the viewpoint of high final efficiency of introduction and high survival rate, mRNA is added to the cells into which the gene is introduced at an amount of 5 to 20 pg/cell. Furthermore, after addition of such a mixed solution, the cells can be cultured according to the manufacturer's instructions. However, from the viewpoint of stabilizing the yield of finally obtained transgenic cells, when the cells into which the gene is introduced are human mesenchymal stem cells or human T cells, it is preferable to culture them for about 1 day (18 hours), and when the cells are human myoblasts, to culture them for about 2 days (42 hours).

For example, when plasmid DNA is introduced into cells using the lipofection method, the plasmid DNA is mixed with a lipofection reagent such as Lipofectamine (Thermo Fisher Scientific) or ViaFect (Promega), or PEI MAX (Polysciences), etc. at an amount of 1 to 100 pg/cell, and added to the cells into which the gene is introduced. Preferably, from the viewpoint of high final efficiency of introduction and high survival rate, plasmid DNA is added to the cells into which the gene is introduced at an amount of 5 to 20 pg/cell.

In the present invention, when the electroporation method is used, a cell solution wherein an electroporation reagent such as R buffer (Thermo Fisher Scientific) is added to cells into which the gene is introduced, is prepared at a concentration of 1×10⁶ to 1×10⁸ cells/mL. Preferably, from the viewpoint of high final efficiency of introduction and survival rate, it is prepared at a concentration of 1×10⁶ to 1×10⁷ cells/mL.

For example, when plasmid DNA is introduced into cells using the electroporation method, the plasmid DNA is mixed with a cell solution at an amount of 1 to 100 pg/cell. Preferably, from the viewpoint of high final efficiency of introduction and survival rate, the plasmid DNA is mixed with the cell solution at an amount of 1 to 50 pg/cell. In addition, for example when electroporation is performed using the Neon Transfection System (Thermo Fisher Scientific), high final efficiency of introduction and survival rate can be achieved by setting Pulse Voltage: 990 to 1650 V, Pulse Width: 10 to 40 ms, and Pulse Number: 1 to 3.

In the present invention, when the polybrene method is used, cells are seeded at about 1,000 to 100,000 cells/cm². Preferably, from the viewpoint of stabilizing the final yield, cells are seeded at about 1,000 to 2,000 cells/cm². When the cells into which the gene is introduced are human mesenchymal stem cells or human myoblasts, the cells are more preferably seeded at 2,000 cells/cm².

For example, when a viral vector is introduced into cells using the polybrene method, the viral vector is added to the cells into which the gene is introduced at an MOI of 0.1 to 30. Preferably, from the viewpoint of high final efficiency of introduction and survival rate, the viral vector is added to the cells into which the gene is introduced at an MOI of 0.1 to 10. In addition, polybrene is added to the cells into which the gene is introduced at an amount of 0 to 20 μg/mL; however, from the viewpoint of high final efficiency of introduction and survival rate, preferably, polybrene is added to such cells at an amount of 0 to 10 μg/mL.

In another aspect, the present invention relates to a method for producing transgenic cells, comprising administering an agent for avoiding the immune response caused by the introduction of genes described above. In yet another aspect, the present invention relates to transgenic cells obtained using the above-mentioned agent for avoiding the immune response caused by the introduction of genes. As described above, the agent of the present invention can increase the yield of cells into which the gene is introduced without limiting the type of cells into which the gene is introduced or the type of the transgene, and regarding the gene and cell in the transgenic cells of the present invention, the genes and cells listed above can be used. The transgenic cells of the present invention can be administered, for example, to a subject in need of mesenchymal stem cells or cells differentiated from mesenchymal stem cells, at a dose of 10,000 to 100,000,000 cells/kg body weight. Examples of such subjects include patients with infectious diseases and neoplasms such as cancer, as well as immune system diseases, circulatory system diseases, central nervous system diseases, motor system diseases, respiratory system diseases, digestive system diseases, urinary system diseases, obstetrics and gynecological diseases, otorhinolaryngological diseases, dental diseases, and skin diseases.

In yet another aspect, the present invention relates to an agent and a method for avoiding an immune response caused by introduction of genes by means of administering a transgene and an NFκB inhibitor or an expression inhibitor of a gene related to innate immunity.

In the present invention, the expression inhibitor of a gene related to innate immunity is any agent that inhibits the expression of a gene related to innate immunity, and examples thereof include, but are not limited to, preferably, siRNA against genes related to innate immunity. In the present invention, the transgene and the NFκB inhibitor or the expression inhibitor of a gene related to innate immunity may be administered simultaneously or separately. In the present invention, the transgene and/or the NFκB inhibitor or the expression inhibitor of a gene related to innate immunity may be administered at the time of implementing the method of introducing the gene into cells, or may be administered in advance prior to implementation. In the present invention, the transgene and/or the NFκB inhibitor or the expression inhibitor of a gene related to innate immunity may be administered so that the concentration of the NFκB inhibitor is 0.025 to 3,000 μg/kg body weight in a subject in need thereof. When the NFκB inhibitor is JSH-23, the transgene and/or the NFκB inhibitor or the expression inhibitor of a gene related to innate immunity may be administered so that the concentration of JSH-23 is 1 to 3 mg/kg body weight in a subject in need thereof. When the NFB inhibitor is calcitriol, the transgene and/or the NFκB inhibitor or the expression inhibitor of a gene related to innate immunity may be administered so that the concentration of calcitriol is 1 to 5,000 IU/kg body weight in a subject in need thereof. In the present invention, the transgene and/or the NFκB inhibitor or the expression inhibitor of a gene related to innate immunity may be administered so that the concentration of the transgene is 10 to 2,000 μg/kg body weight in a subject in need thereof.

In yet another aspect, the present invention relates to a method for avoiding immune responses caused by introduction of genes in cells into which the gene is introduced. The method for suppressing innate immunity in the present invention is as described above. The present invention may be in vitro or may be in vivo. In one embodiment, the yield of transgenic cells is increased by performing the method of the present invention in vitro.

### EXAMPLES

Hereinafter, the present invention will be explained in more detail based on examples, but it goes without saying that the present invention is not limited to these examples.

In the following examples, the following reagents, culture media, equipment, and the like were used.

### Reagent

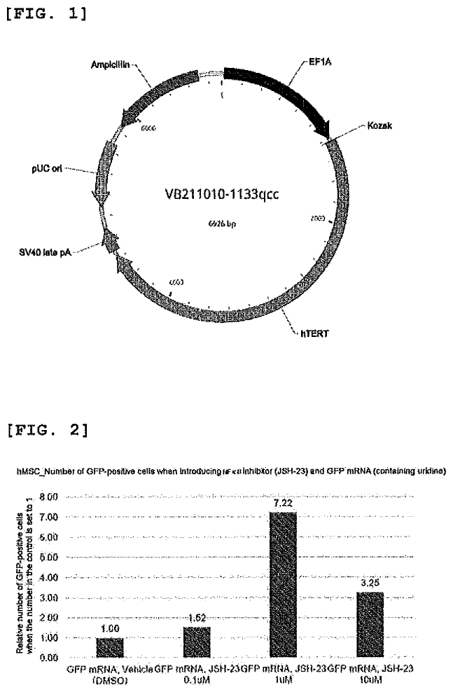

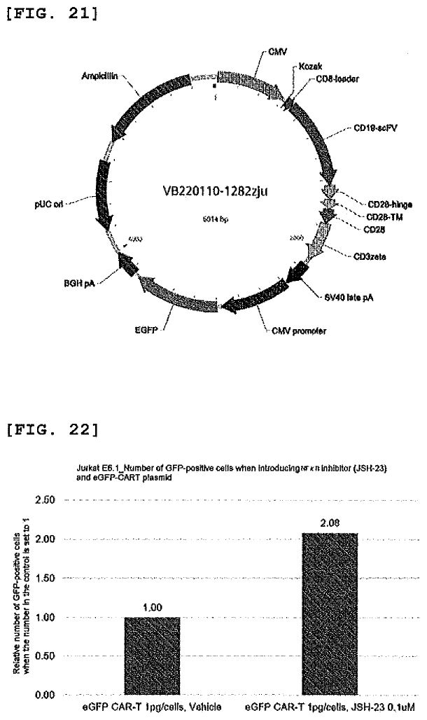

Human mesenchymal stem cells (Lonza, product number: PT-2501)
Human myoblasts (Lonza, product number: CC-2580)
Human leukemia T cells (Jurkat E6.1) (KAC, model number: EC88042803-F0)
GFP mRNA containing uridine (SEQ ID NO: 1)
GFP mRNA containing pseudouridine instead of uridine (all uridine in uridine-containing GFP mRNA is replaced with pseudouridine (TriLink, product number: N-1019-10))
TERT mRNA containing uridine (SEQ ID NO: 2)
TERT mRNA containing pseudouridine instead of uridine (all uridine in uridine-containing TERT mRNA is replaced with pseudouridine)
GFP viral vector (VectorBuilder, Control Vector ID: VB160109-10005)
TERT plasmid DNA (shown in FIG. 1)
CAR-T plasmid DNA (shown in FIG. 21)
αMEM (NACALAI TESQUE, product number: 21444-05)
MCDB131 (GIBCO, product number: 10372-019)
RPMI1640 medium (NACALAI TESQUE, product number: 30264-85)
Fetal bovine serum (hereinafter referred to as serum) (GIBCO, product number: 26140-079)
JSH-23 (abcam, product number: ab144824)
Calcitriol (cayman chemical, product number: 71820)
Vitamin B6 (Fujifilm Wako Pure Chemical, product number: 010-13931)
Vitamin C (NACALAI TESQUE, product number: 1170434)
Vitamin E (NACALAI TESQUE, product number: 3411544)
Rolipram (abcam, product number: ab120031)
BAY11 (abcam, product number: ab141228)
Dexamethasone (Aspen Japan, product number: 19206C1)
6-Amino-4-(4-phenoxyphenethylamino)quinazoline (Tokyo Chemical Industry Co., Ltd., product number: Q0109)
OptiMEM (GIBCO, product number: 31985-062)
Lipofectamine RNAiMAX (Thermo Fisher Scientific, product number: 13778075)
Lipofectamine 2000 (Thermo Fisher Scientific, product number: 11668027)
PEI MAX (Polysciences, product number: 24765-100)
Cell detachment solution (GIBCO, product number: 2605-028)
Polybrene solution (Vector Builder, product number: PL0001)
PureLink™ RNA Mini Kit (Thermo Fischer Scientific, product number: 12183018A)
SuperScript™ III Reverse Transcriptase (Thermo Fisher Scientific, product number: 18080044)

TNF-α primer (forward):
(SEQ ID NO: 4)
ATCAATCGGCCCGACTATCTC
TNF-α primer (reverse):
(SEQ ID NO: 5)
GCAATGATCCAAAGTAGACC
ß-actin primer (forward):
(SEQ ID NO: 6)
AGAAATCTGGCACCACACC
ß-actin primer (reverse):
(SEQ ID NO: 7)
AGAGGCGTACAGGGATAGCA
IFN-α primer (forward):
(SEQ ID NO: 8)
TTCTCTGGGCTGTGATCTGC
IFN-α primer (reverse):
(SEQ ID NO: 9)
CTGTCCTTCAGGCAGGAGAAA

PowerTrack™ SYBR Green Master Mix (Thermo Fisher Scientific, product number: A46012)
siRNA (TNF-α) (Ambion, product number: s14247, s14248)
siRNA (IFN-α) (Ambion, product number: s226340, s226337)
Negative Control siRNA (Negative Control No. 1) (Ambion,
product number: 4390843)
Culture medium
Basal medium (for human mesenchymal stem cells): αMEM+10% serum
Basal medium (for human myoblasts): MCDB131+10% serum Equipment
CO2 incubator (astec, model number: CPI-165R)
Centrifuge (KUBOTA, model number: 5911)
Flow cytometer (SYSMEX, model number: RF-500)

### Example 1. Yield Improvement Using JSH-23 in Introduction of GFP mRNA into Human Mesenchymal Stem Cells (hMSCs) and Human Myoblasts (hSMMs)

Human mesenchymal stem cells were seeded in a 6-well plate to be about 10,000 cells/cm². Human myoblasts were seeded in another 6-well plate to be about 20,000 cells/cm². As a culture solution, a basal medium for each cell supplemented with 0.1, 1, or 10 μM of JSH-23 was used. As a control, a basal medium for each cell supplemented with 0.1% (v/v) DMSO instead of JSH-23 was used as a culture solution.

A lipofection solution was prepared by adding Lipofectamine RNAiMAX and fluorescent protein GFP mRNA to OptiMEM. As the GFP mRNA, that containing uridine or that containing pseudouridine instead of uridine was used. In addition, for those containing pseudouridine, a basal medium for each cell supplemented with 1 μM of JSH-23 was used, and as a control, a basal medium for each cell supplemented with 0.1% (v/v) DMSO instead of JSH-23 was used as a culture solution. It was prepared so that the amount of mRNA was 10 pg/cell for human mesenchymal stem cells and 5 pg/cell for human myoblasts. The prepared lipofection solution was mixed well according to the manufacturer's recommended protocol and allowed to stand at room temperature for about 15 minutes.

Next, each lipofection solution was added to the plate seeded with human mesenchymal stem cells or human myoblasts, and cultured in a CO2 incubator at 37° C. for about 1 day (18 hours) (human mesenchymal stem cells) or for 2 days (42 hours) (human myoblasts). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells or human myoblasts, and cultured in a CO2 incubator at 37° C. for about 1 day (18 hours) (mesenchymal stem cells) or about 2 days (42 hours) (human myoblasts). These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive rate. The results are shown in FIGS. 2 to 5.

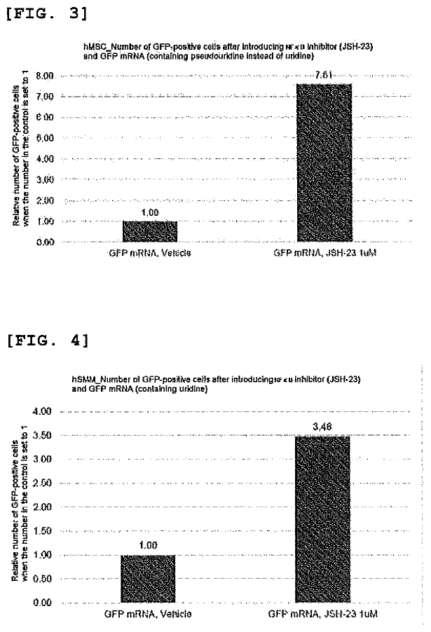

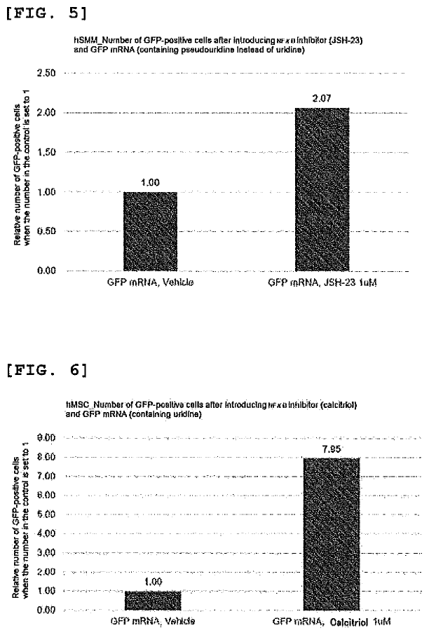

FIG. 2 is a graph showing the number of GFP-positive cells when GFP mRNA containing uridine was introduced into human mesenchymal stem cells using each culture solution. FIG. 3 is a graph showing the number of GFP-positive cells when GFP mRNA containing pseudouridine instead of uridine was introduced into human mesenchymal stem cells using each culture solution. FIG. 4 is a graph showing the number of GFP-positive cells when GFP mRNA containing uridine was introduced into human myoblasts using each culture solution. FIG. 5 is a graph showing the number of GFP-positive cells when GFP mRNA containing pseudouridine instead of uridine was introduced into human myoblasts using each culture solution. In each figure, the vertical axis shows the respective relative number of cells when the number of cells in the control is set to 1. From FIGS. 2 and 3, when the GFP mRNA containing uridine was used in human mesenchymal stem cells, it was confirmed that the number of GFP-positive cells increased by adding JSH-23, and the number was maximally increased with JSH-23 of 1 μM. Under the conditions tested, maximally a yield of 7 times or more could be secured, i.e., starting from the same number of cells, 7 times or more transgenic cells compared to the control could be secured. In addition, when the GFP mRNA containing pseudouridine instead of uridine was used, it was also confirmed that the number of GFP-positive cells increased by adding JSH-23. Under the tested conditions, maximally a yield of 7 times or more could be secured. From FIGS. 4 and 5, in human myoblasts, regardless of whether GFP mRNA containing uridine or GFP mRNA containing pseudouridine instead of uridine is used, it was confirmed that the number of GFP-positive cells increased by adding JSH-23. Under the conditions tested, for GFP mRNA containing uridine, a yield of 3 times or more could be secured, i.e., starting from the same number of cells, 3 times or more transgenic cells compared to the control could be secured. In the case of GFP mRNA containing pseudouridine instead of uridine, a yield of 2 times or more could be secured.

### Example 2. Yield Improvement Using Calcitriol in Introduction of GFP mRNA into Human Mesenchymal Stem Cells (hMSCs) and Human Myoblasts (hSMMs)

Human mesenchymal stem cells were seeded in a 6-well plate to be about 10,000 cells/cm². Human myoblasts were seeded in another 6-well plate to be about 20,000 cells/cm². As a culture solution, a basal medium for each cell supplemented with 1 μM of calcitriol was used. As a control, a basal medium for each cell supplemented with 0.1% (v/v) DMSO instead of calcitriol was used as a culture solution.

A lipofection solution was prepared by adding Lipofectamine RNAiMAX and fluorescent protein GFP mRNA to OptiMEM. As the GFP mRNA, that containing uridine, or that containing pseudouridine instead of uridine was used. It was prepared so that the amount of mRNA was 10 pg/cell for human mesenchymal stem cells and 5 pg/cell for human myoblasts. The prepared lipofection solution was mixed well according to the manufacturer's recommended protocol and allowed to stand at room temperature for about 15 minutes.

Next, each lipofection solution was added to the plate seeded with human mesenchymal stem cells or human myoblasts, and cultured in a CO2 incubator at 37° C. for about 1 day (18 hours) (human mesenchymal stem cells) or for 2 days (42 hours) (human myoblasts). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells or human myoblasts, and cultured in a CO2 incubator at 37° C. for about 1 day (18 hours) (mesenchymal stem cells) or about 2 days (42 hours) (human myoblasts). These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive rate. The results are shown in FIGS. 6 to 9.

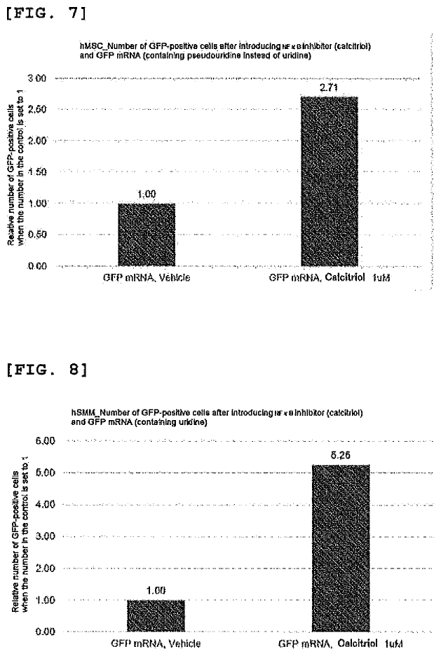

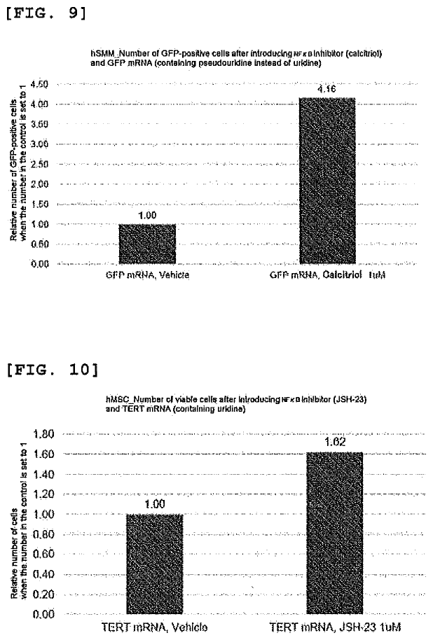

FIG. 6 is a graph showing the number of GFP-positive cells when GFP mRNA containing uridine was introduced into human mesenchymal stem cells using each culture solution. FIG. 7 is a graph showing the number of GFP-positive cells when GFP mRNA containing pseudouridine instead of uridine was introduced into human mesenchymal stem cells using each culture solution. FIG. 8 is a graph showing the number of GFP-positive cells when GFP mRNA containing uridine was introduced into human myoblasts using each culture solution. FIG. 9 is a graph showing the number of GFP-positive cells when GFP mRNA containing pseudouridine instead of uridine was introduced into human myoblasts using each culture solution. In each figure, the vertical axis shows the respective relative number of cells when the number of cells in the control is set to 1. From the above, in both human mesenchymal stem cells and human myoblasts, regardless of whether GFP mRNA containing uridine or GFP mRNA containing pseudouridine instead of uridine is used, it was confirmed that the number of GFP-positive cells increased by adding calcitriol. Under the conditions tested, for the introduction of GFP mRNA containing uridine into human myoblasts, a yield of 7 times or more, i.e., starting from the same number of cells, 7 times or more transgenic cells compared to the control could be secured. In the introduction of GFP mRNA containing pseudouridine instead of uridine into human mesenchymal stem cells, a yield of 2.7 times or more could be secured. In addition, under the conditions tested, for the introduction of GFP mRNA containing uridine into human myoblasts, a yield of 5 times or more, i.e., starting from the same number of cells, 5 times or more transgenic cells compared to the control could be secured. In the introduction of GFP mRNA containing pseudouridine instead of uridine into human myoblasts, a yield of 4 times or more could be secured.

### Example 3. Yield Improvement Using JSH-23 in Introduction of TERT mRNA into Human Mesenchymal Stem Cells (hMSCs) and Human Myoblasts (hSMMs)

Human mesenchymal stem cells were seeded in a 6-well plate to be about 10,000 cells/cm². Human myoblasts were seeded in another 6-well plate to be about 20,000 cells/cm². As a culture solution, a basal medium for each cell supplemented with 1 μM of JSH-23 was used. As a control, a basal medium for each cell supplemented with 0.1% (v/v) DMSO instead of JSH-23 was used as a culture solution.

A lipofection solution was prepared by adding Lipofectamine RNAiMAX and TERT mRNA to OptiMEM. As the TERT mRNA, that containing uridine, or that containing pseudouridine instead of uridine was used. It was prepared so that the amount of mRNA was 10 pg/cell for human mesenchymal stem cells and 5 pg/cell for human myoblasts. The prepared lipofection solution was mixed well according to the manufacturer's recommended protocol and allowed to stand at room temperature for about 15 minutes.

Next, each lipofection solution was added to the plate seeded with human mesenchymal stem cells or human myoblasts, and cultured in a CO2 incubator at 37° C. for about 1 day (18 hours) (human mesenchymal stem cells) or for 2 days (42 hours) (human myoblasts). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells or human myoblasts, and cultured in a CO2 incubator at 37° C. for about 1 day (18 hours) (mesenchymal stem cells) or about 2 days (42 hours) (human myoblasts). These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. The collected cells were counted using a hemocytometer. The results are shown in FIGS. 10 to 13.

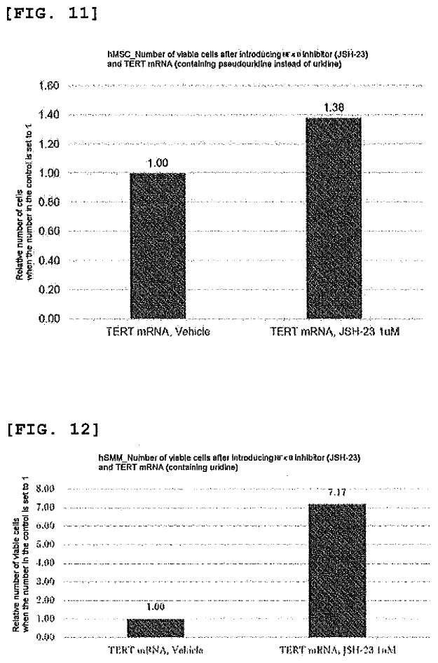

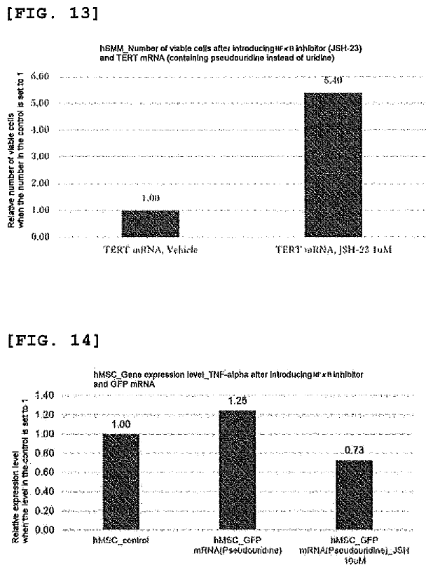

FIG. 10 is a graph showing the number of viable cells when TERT mRNA containing uridine was introduced into human mesenchymal stem cells using each culture solution. FIG. 11 is a graph showing the number of viable cells when TERT mRNA containing pseudouridine instead of uridine was introduced into human mesenchymal stem cells using each culture solution. FIG. 12 is a graph showing the number of viable cells when TERT mRNA containing uridine was introduced into human myoblasts using each culture solution. FIG. 13 is a graph showing the number of viable cells when TERT mRNA containing pseudouridine instead of uridine was introduced into human myoblasts using each culture solution. In each figure, the vertical axis shows the respective relative number of cells when the number of cells in the control is set to 1. From the above, in both human mesenchymal stem cells and human myoblasts, regardless of whether TERT mRNA containing uridine or TERT mRNA containing pseudouridine instead of uridine is used, it was confirmed that the number of viable cells increased by adding JSH-23. Under the conditions tested, for the introduction of TERT mRNA containing uridine into human mesenchymal stem cells, a yield of 1.5 times or more, i.e., starting from the same number of cells, 1.5 times or more transgenic cells compared to the control could be secured. In the introduction of TERT mRNA containing pseudouridine instead of uridine into human mesenchymal stem cells, a yield of 1.3 times or more could be secured. In addition, under the conditions tested, for the introduction of TERT mRNA containing uridine into human myoblasts, a yield of 7 times or more, i.e., starting from the same number of cells, 7 times or more transgenic cells compared to the control could be secured. In the introduction of TERT mRNA containing pseudouridine instead of uridine into human myoblasts, a yield of 5 times or more could be secured.

### Example 4. Enhancement of Immunosuppression

Reduction of TNF-α expression level using JSH-23 in introduction of GFP mRNA into human mesenchymal stem cells (hMSCs) Human mesenchymal stem cells were seeded in a 6-well plate to be about 10,000 cells/cm². As a culture solution, αMEM (+10% serum) supplemented with 10 μM of JSH-23 was used. As a control, αMEM (+10% serum) supplemented with 0.1% (v/v) DMSO instead of JSH-23 was used as a culture solution.

A lipofection solution was prepared by adding Lipofectamine RNAiMAX and fluorescent protein GFP mRNA to OptiMEM. The GFP mRNA containing pseudouridine instead of uridine was used. It was prepared so that the amount of mRNA was 10 pg/cell. The prepared lipofection solution was mixed well according to the manufacturer's recommended protocol and allowed to stand at room temperature for about 15 minutes.

Next, each lipofection solution was added to the plate seeded with human mesenchymal stem cells or human myoblasts, and cultured in a CO2 incubator at 37° C. for about 1 day (18 hours). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells or human myoblasts, and cultured in a CO2 incubator at 37° C. for about 1 day (18 hours). These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. Using the PureLink RNA Mini kit according to the manufacturer's protocol, the collected cells were lysed and RNA was extracted and purified.

cDNA was synthesized from the purified RNA using SuperScript™ III Reverse Transcriptase according to the manufacturer's protocol. qPCR was performed using synthesized cDNA and PCR primers and PowerTrack™ SYBR Green Master Mix. The measured Ct value of TNF-α was normalized with the Ct value of β-actin, a housekeeping gene. The results are shown in FIG. 14.

FIG. 14 is a graph showing the expression level in cells introduced with mRNA using pseudouridine, and cells introduced with mRNA using pseudouridine using JSH-23, when the expression level of TNF-α in the control is set to 1. The purpose of using pseudouridine during introduction of mRNA is to reduce the activation of TLR (Toll-like receptor), thereby reducing the activation of NFκB and TNF-α downstream of the signal, and preventing cell death. However, as shown in FIG. 14, when only pseudouridine was used, the expression level of TNF-α increased. On the other hand, it was confirmed that the use of JSH-23 further reduced the expression level of TNF-α compared to the case in which pseudouridine was used.

### Example 5. Yield Improvement Using JSH-23 in Introduction of TERT Plasmid into Human Mesenchymal Stem Cells (hMSCs)

Human mesenchymal stem cells were seeded in a 6-well plate to be about 10,000 cells/cm². As a culture solution, αMEM (+10% serum) supplemented with 1 μM of JSH-23 was used. As a control, αMEM (+10% serum) supplemented with 0.1% (v/v) DMSO instead of JSH-23 was used as a culture solution.

A lipofection solution was prepared by adding Lipofectamine RNAiMAX and TERT plasmid DNA to OptiMEM. The amount of plasmid DNA was prepared to be 10 pg/cell.

Next, the lipofection solution was added to the plate seeded with human mesenchymal stem cells, and cultured in a CO2 incubator at 37° C. for 18 hours. After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells, and cultured in a CO2 incubator at 37° C. for 18 hours. These steps were repeated so that the introduction of genes was performed a total of 4 times.

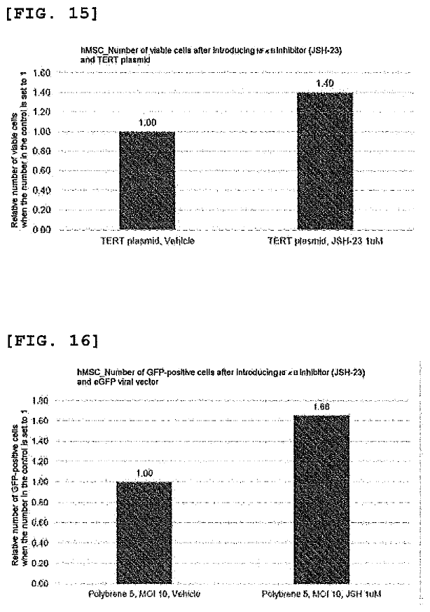

Twenty-four hours after the final introduction of genes, the cells were detached with a cell detachment solution, centrifuged at 600×g for 5 minutes using a centrifuge, to collect the cells. The collected cells were counted using a hemocytometer. The results are shown in FIG. 15.

FIG. 15 is a graph showing the number of viable cells when TERT plasmid was introduced into human mesenchymal stem cells using each culture solution. The vertical axis shows the relative number of cells when the number of cells in the control is set to 1. From the above, it was confirmed that the number of viable cells increased by adding JSH-23. Under the conditions tested, 1.4 times or more cells, i.e., starting from the same number of cells, 1.4 times or more transgenic cells compared to the control could be secured.

### Example 6. Yield Improvement Using JSH-23 in Introduction of GFP Viral Vector into Human Mesenchymal Stem Cells (hMSCs) and Human Myoblasts (hSMMs)

Human mesenchymal stem cells were seeded in a 6-well plate to be about 2,000 cells/cm². Human myoblasts were seeded in another 6-well plate to be about 2,000 cells/cm². As a culture solution, a basal medium for each cell supplemented with JSH-23 was used. JSH-23 was added at 1 μM to the culture solution used for human mesenchymal stem cells, and at 0.1 μM to the culture solution used for human myoblasts. As a control, a basal medium, i.e., αMEM (+10% serum) for human mesenchymal stem cells and MCDB131 (+10% serum) for human myoblasts, to which 0.1% (v/v) DMSO instead of JSH-23 was added was used as a culture solution.

A mixed solution was prepared by mixing GFP viral vector and a polybrene solution. The amount of GFP viral vector was prepared to be MOI of 10, and the amount of polybrene was prepared to be 5 μg/mL.

Next, the above mixed solution was added to the plate seeded with human mesenchymal stem cells or human myoblasts, and cultured in a CO2 incubator at 37° C. for 24 hours. After culturing, regarding the respective cells, the culture solution was replaced with that does not contain the GFP viral vector, polybrene, or JSH-23 (virus-free culture solution).

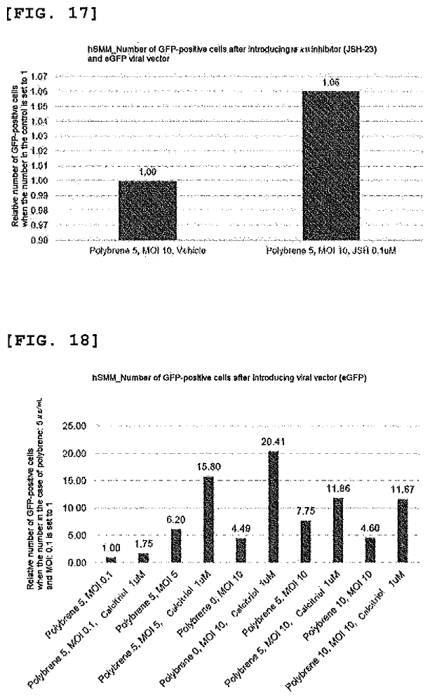

Four days after the addition of the virus-free culture solution, the cells were detached with a cell detachment solution and centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIGS. 16 and 17.

FIG. 16 is a graph showing the number of GFP-positive cells when GFP viral vector was introduced into human mesenchymal stem cells using each culture solution. FIG. 17 is a graph showing the number of GFP-positive cells in each culture solution when GFP viral vector was introduced into human myoblasts. The vertical axis of each figure shows the relative number of cells when the number of cells in the control is set to 1. From the above, it was confirmed that in both human mesenchymal stem cells and human myoblasts, the number of GFP-positive cells increased by the addition of JSH-23.

### Example 7. Yield Improvement Using Calcitriol in Introduction of GFP Viral Vector into Human Myoblasts (hSMMs)

Human myoblasts were seeded in a 6-well plate to be about 2,000 cells/cm². As a culture solution, MCDB131 (+10% serum) supplemented with 1 μM of calcitriol was used. As a control, MCDB131 (+10% serum) supplemented with 0.1% (v/v) DMSO instead of calcitriol was used as a culture solution.

GFP viral vector and a polybrene solution were mixed. Each of the mixed solutions was prepared so that the amount of the viral vector was MOI of 0.1, 5, or 10, and the amount of polybrene was 0, 5, or 10 μg/mL.

Next, the above mixed solution was added to the plate seeded with human myoblasts, and cultured in a CO2 incubator at 37° C. for 24 hours. After culturing, the culture solution was replaced with that does not contain the GFP viral vector, polybrene, and JSH-23 (virus-free culture solution).

Four days after the addition of the virus-free culture solution, the cells were detached with a cell detachment solution and centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIG. 18.

FIG. 18 is a graph showing the number of GFP-positive cells when GFP viral vector was introduced into human myoblasts using each mixed solution. The vertical axis shows the relative number of cells in each case, when the number of cells in the mixed solution comprising polybrene (5 pg/mL) and viral vector (MOI of 0.1) is set to 1. From the above, it was confirmed that the number of GFP-positive cells increased by adding calcitriol. It was also confirmed that the number of GFP-positive cells increased with the addition of calcitriol for any amount of viral vector or polybrene. Depending on the transduction conditions, the increase was about 4.5 times.

### Example 8. Improvement of the Amount of Transgene Using JSH-23 in Introduction of GFP mRNA into Human Mesenchymal Stem Cells (hMSCs)

Human mesenchymal stem cells were seeded in a 6-well plate to be about 10,000 cells/cm². As a culture solution, αMEM (+10% serum) supplemented with 1 μM of JSH-23 or αMEM (+10% serum) supplemented with 0.1% (v/v) DMSO instead of JSH-23 was used.

A lipofection solution was prepared by adding Lipofectamine RNAiMAX and fluorescent protein GFP mRNA to OptiMEM. It was prepared so that the amount of mRNA was 10 pg/cell or 20 pg/cell. The prepared lipofection solution was mixed well according to the manufacturer's recommended protocol and allowed to stand at room temperature for about 15 minutes.

Next, a lipofection solution prepared so that the amount of mRNA was 20 pg/cell was added to a plate that used, as a culture solution, αMEM (+10% serum) supplemented with DMSO or JSH-23, respectively. As a control, a lipofection solution prepared so that the amount of mRNA was 10 pg/cell was added to a plate that used αMEM (+10% serum) supplemented with DMSO as a culture solution. Subsequently, after culturing in a CO2 incubator at 37° C. for 18 hours, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells, and cultured in a CO2 incubator at 37° C. for 18 hours. These steps were repeated so that the introduction of genes was performed a total of 4 times.

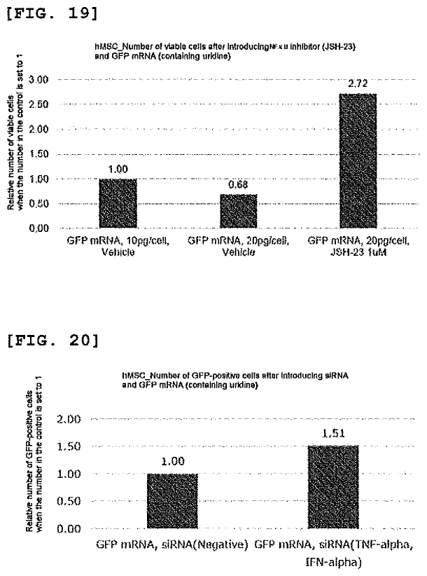

Twenty-four hours after the final introduction of genes, the cells were detached with a cell detachment solution, centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIG. 19.

FIG. 19 is a graph showing the number of GFP-positive cells when GFP mRNA was introduced into human mesenchymal stem cells under each condition. The vertical axis shows the relative number of cells when the number of cells in the control is set to 1. Increasing the amount of GFP mRNA in the lipofection solution from 10 pg/cell to 20 pg/cell decreased the number of GFP-positive cells; however, even when the amount of GFP mRNA was 20 pg/cell, it was confirmed that the number of GFP-positive cells increased by adding JSH-23.

In general, increasing the amount of transgene increases the number of dead cells; however, by using the agent of the present invention, introduction of genes at high concentration is possible while maintaining a high survival rate. Therefore, it is considered that the efficiency of introduction of genes into cells can be further improved.

### Example 9. Innate Immune Evasion by siRNA (TNF-α, IFN-α)

Human mesenchymal stem cells were seeded in a 6-well plate to be about 10,000 cells/cm².

A lipofection solution was prepared by adding Lipofectamine RNAiMAX and fluorescent protein GFP mRNA to OptiMEM. It was prepared so that the amount of mRNA was 10 pg/cell. TNF-α and IFN-α siRNA were added to the prepared lipofection solution at 5 nM. As a control, Negative Control siRNA was added at 5 nM. The lipofection solution supplemented with siRNA was mixed well according to the manufacturer's recommended protocols, and allowed to stand at room temperature for about 15 minutes.

Next, each lipofection solution was added to the plate seeded with human mesenchymal stem cells, and cultured in a CO2 incubator at 37° C. for 18 hours. After culturing, all the culture solution comprising the lipofection solution was removed, and replaced with a new culture solution having the same composition as before the addition of the lipofection solution.

After another 6 hours, the above lipofection solution was newly prepared, added again to the plate seeded with human mesenchymal stem cells, and cultured in a CO2 incubator at 37° C. for 18 hours. These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIG. 20.

FIG. 20 is a graph showing the number of GFP-positive cells when GFP mRNA was introduced into human mesenchymal stem cells using a lipofection solution supplemented with each siRNA. The vertical axis shows the relative number of cells when the number of cells in the control is set to 1. From the above, it was confirmed that in human mesenchymal stem cells, the number of GFP-positive cells increased by using TNF-α and IFN-α siRNA during lipofection. Under the conditions tested, a yield of 1.5 times or more, i.e., starting from the same number of cells, 1.5 times or more transgenic cells compared to the control could be secured.

### Example 10. Yield Improvement Using JSH-23 in Introduction Of CAR-T Plasmid Genes into Human Leukemia T Cells (Jurkat E6.1)

Human leukemia T cells (Jurkat E6.1) were seeded in a 6-well plate to be about 10,000 cells/cm². As a culture solution, RPMI1640 medium comprising 10% bovine serum was used. To this, 0.1 μM of JSH-23 was added, or for comparison, 0.1% (v/v) DMSO instead of JSH-23 was added and used.

A lipofection solution was prepared by adding Lipofectamine 2000 and GFP-tagged CAR-T plasmid to OptiMEM. The amount of CAR-T plasmid was prepared to be 1 pg/cell. The prepared lipofection solution was mixed well according to the manufacturer's recommended protocol and allowed to stand at room temperature for about 15 minutes.

Next, the lipofection solution was added to the plate seeded with human leukemia T cells, and cultured in a CO2 incubator at 37° C. for about 1 day (18 hours). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human leukemia T cells, and cultured in a CO2 incubator at 37° C. for about 1 day (18 hours). These steps were repeated so that the introduction of genes was performed a total of 4 times.

Twenty-four hours after the final introduction of genes, the cells were collected and centrifuged at 800 rpm for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage.

The results are shown in FIG. 22.

FIG. 22 is a graph showing the number of GFP-positive cells when a GFP-tagged CAR-T plasmid gene was introduced into human leukemia T cells using each culture solution. The vertical axis shows the relative number of cells when the number of cells in the control is set to 1. From the above, it was confirmed that the number of GFP-positive cells increased by adding JSH-23. Under the conditions tested, 2 times or more cells, i.e., starting from the same number of cells, 2 times or more transgenic cells compared to the control could be secured.

### Example 11. Evaluation of Immunosuppression when Introducing Plasmid Genes

Human leukemia T cells (Jurkat E6.1) were seeded in a 6-well plate to be about 10,000 cells/cm². As a culture solution, RPMI1640 medium comprising 10% bovine serum was used. To this, 0.1 μM of JSH-23 was added, or for comparison, 0.1% (v/v) DMSO instead of JSH-23 was added and used.

A lipofection solution was prepared by adding Lipofectamine 2000 and GFP-tagged CAR-T plasmid to OptiMEM. The amount of plasmid was prepared to be 1 pg/cell. The prepared lipofection solution was mixed well according to the manufacturer's recommended protocol and allowed to stand at room temperature for about 15 minutes.

Next, the lipofection solution was added to the plate seeded with human leukemia T cells, and cultured in a CO2 incubator at 37° C. for about 1 day (18 hours). After culturing, all the culture solution comprising the lipofection solution was removed and replaced with a new culture solution having the same composition as before the addition of the lipofection solution.

After another 6 hours, the above lipofection solution was newly prepared, added again to the plate seeded with human leukemia T cells, and cultured in a CO2 incubator at 37° C. for about 1 day (18 hours). These steps were repeated so that the introduction of genes was performed a total of 4 times.

Twenty-four hours after the final introduction of genes, the cells were collected and centrifuged at 800 rpm for 5 minutes using a centrifuge, to collect the cells. Using the PureLink RNA Mini kit according to the manufacturer's protocol, the collected cells were lysed and RNA was extracted and purified.

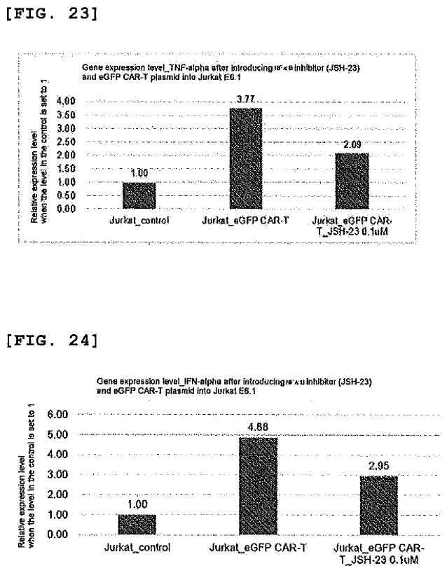

cDNA was synthesized from the purified RNA using SuperScript™ III Reverse Transcriptase according to the manufacturer's protocol. qPCR was performed using the synthesized cDNA and PCR primers as well as PowerTrack™ SYBR Green Master Mix. The measured Ct value of TNF-α was normalized with the Ct value of β-actin, a housekeeping gene. The results are shown in FIGS. 23 and 24.

FIG. 23 is a graph showing the expression level of TNF-α in the cells introduced with GFP-tagged CAR-T plasmid, and in the cells introduced with CAR-T plasmid using JSH-23. The vertical axis shows the relative expression level in each of the cells, when the expression level of TNF-α in the control human leukemia T cells without introduction of plasmid is set to 1. FIG. 24 is a graph showing the expression level of IFN-α in the cells introduced with GFP-tagged CAR-T plasmid, and in the cells introduced with CAR-T plasmid using JSH-23. The vertical axis shows the relative expression level in each of the cells, when the expression level of IFN-α in the control human leukemia T cells without introduction of plasmid is set to 1. From the above, it was confirmed that when CAR-T plasmid was introduced using JSH-23, the increase in the expression levels of both TNF-α and IFN-α genes reduced compared to when CAR-T plasmid was introduced without using JSH-23.

TNF-α and IFN-α are indicators of immune system activity, and their expression levels increase as an immune response when a foreign gene is incorporated into cells. Since excessive immune response leads to cell death, suppression of the immune response has been a challenge for the generation of foreign transgenic cells. However, as shown in FIGS. 23 and 24, it was found that by using JSH-23, the increase in the expression levels of TNF-α and IFN-α can be reduced, that is, the immune response can be suppressed. This suppression of immune response is considered to lead to improved yield of transgenic cells.

### Example 12. Investigation of Yield Improvement Using BAY11, Dexamethasone, and 6-amino-4-(4-phenoxyphenethylamino)quinazoline

Improvement in the yield of transgenic cells when using an NFκB inhibitor other than the NFκB nuclear translocation inhibitor of the present invention was investigated. As the NFκB inhibitors, BAY11 (IKK inhibitor), dexamethasone (IκB degradation inhibitor), and 6-amino-4-(4-phenoxyphenethylamino)quinazoline (p65 acetylation inhibitor) were used to perform introduction of genes. For comparison, introduction of gene was performed under the same conditions using JSH-23, an NFκB nuclear translocation inhibitor, instead of the above-mentioned NFκB inhibitors. The specific procedure is as follows.

Human mesenchymal stem cells were seeded in a 6-well plate to be about 10,000 cells/cm². As a culture solution, a basal medium supplemented with BAY11 (hereinafter referred to as BAY) and dexamethasone (hereinafter referred to as Dex) at 0.01 μM, 0.1 μM, 1 μM, respectively, or with 6-amino-4-(4-phenoxyphenethylamino)quinazoline (hereinafter referred to as QNZ) at 1.5 nM, 15 nM, 150 nM was used. For comparison, a basal medium supplemented with 1 μM of JSH-23 instead of each compound was used. As a control, a basal medium for each cell supplemented with 0.1% (v/v) DMSO instead of JSH-23 was used as a culture solution.

A lipofection solution was prepared by adding Lipofectamine RNAiMAX and fluorescent protein GFP mRNA to OptiMEM. It was prepared so that the amount of mRNA was 10 pg/cell. The prepared lipofection solution was mixed well according to the manufacturer's recommended protocol and allowed to stand at room temperature for about 15 minutes.

Next, each lipofection solution was added to the plate seeded with human mesenchymal stem cells, and cultured in a CO2 incubator at 37° C. for 18 hours. After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells, and cultured in a CO2 incubator at 37° C. for 18 hours. These steps were repeated so that the introduction of genes was performed a total of 4 times.

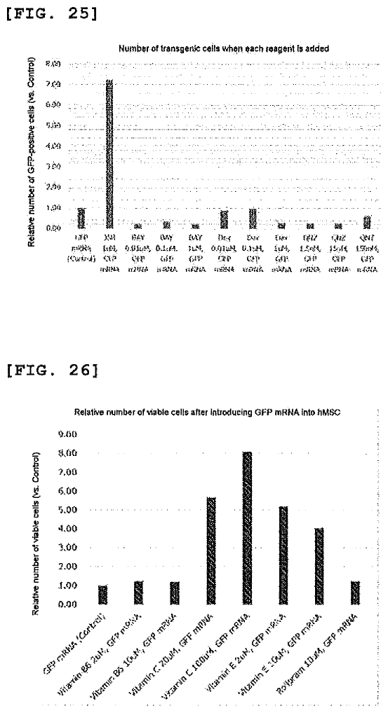

Twenty-four hours after the final introduction of genes, the cells were detached with a cell detachment solution, centrifuged at 600×g for 5 minutes using a centrifuge, to collect the cells. The collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIG. 25.

FIG. 25 is a graph showing the number of GFP-positive cells when GFP mRNA was introduced into human mesenchymal stem cells using each culture solution. In the figure, the vertical axis shows the respective relative number of cells when the number of cells in the control is set to 1. Under the tested conditions, 7 times or more transgenic cells could be secured when using JSH-23; whereas with BAY it was about 0.2 to 0.4 times, with Dex it was about 0.3 to 1.0 times, and with QNZ it was about 0.2 to 0.6 times. From FIG. 25, it was confirmed that the use of JSH-23 as an NFκB nuclear translocation inhibitor is particularly preferable in improving the yield of transgenic cells.

### Example 13. Investigation of Yield Improvement Using NFκB Nuclear Translocation Inhibitor

Improvement in the yield of transgenic cells was investigated when using the NFκB nuclear translocation inhibitor of the present invention other than JSH-23 and calcitriol. Introduction of genes was performed using vitamin B6, vitamin C, vitamin E, and rolipram as an NFκB nuclear translocation inhibitor other than JSH-23 and calcitriol. The specific procedure is as follows.

Human mesenchymal stem cells were seeded in a 6-well plate to be about 10,000 cells/cm². As a culture solution, a basal medium supplemented with vitamin B6 and vitamin E at 2 μM and 10 μM, vitamin C at 20 μM and 100 μM, and rolipram at 10 μM was used. As a control, the basal medium for each cell supplemented with 0.1% (v/v) DMSO instead of each compound was used as a culture solution.

A lipofection solution was prepared by adding PEIMAX and fluorescent protein GFP mRNA to OptiMEM. It was prepared so that the amount of mRNA was 10 pg/cell. The prepared lipofection solution was mixed well according to the manufacturer's recommended protocol and allowed to stand at room temperature for about 15 minutes.

Next, each lipofection solution was added to the plate seeded with human mesenchymal stem cells, and cultured in a CO2 incubator at 37° C. for 18 hours. After culturing, all the culture solution comprising the lipofection solution was removed and replaced with a new culture solution having the same composition as before the addition of the lipofection solution.

After another 6 hours, the above lipofection solution was newly prepared, added again to the plate seeded with human mesenchymal stem cells, and cultured in a CO2 incubator at 37° C. for 18 hours. These steps were repeated so that the introduction of genes was performed a total of 4 times.

Twenty-four hours after the final introduction of genes, the cells were detached with a cell detachment solution, centrifuged at 600×g for 5 minutes using a centrifuge, to collect the cells. In addition, the collected cells were counted using a hemocytometer. Furthermore, the GFP-positive percentage of the collected cells was measured using a flow cytometer. The yield of the number of GFP-positive cells was calculated from the number of cells counted and the GFP-positive percentage. The results are shown in FIG. 26.

FIG. 26 is a graph showing the number of viable cells after introduction of GFP, when GFP mRNA was introduced into human mesenchymal stem cells using each culture solution. In the figure, the vertical axis shows the respective relative number of cells when the number of cells in the control is set to 1. Under the tested conditions, after introduction of genes, it was possible to secure about 1.2 times as many viable cells when using vitamin B6, about 5.6 to 8.2 times when using vitamin C, about 4.0 to 5.2 times when using vitamin E, and about 1.3 times when using rolipram.

1. An agent used to increase the number of transgenic cells, wherein the agent comprises:
(i) an NFκB nuclear translocation inhibitor, or
(ii) an expression inhibitor of a gene related to innate immunity;
and suppresses the innate immunity of cells against transgenes.

2. The agent according to claim 1, wherein the NFB nuclear translocation inhibitor is JSH-21, JSH-23, vitamin E, calcitriol, calcifediol, vitamin D, vitamin C, vitamin B6, rolipram, SN50, or a derivative thereof.

3. The agent according to claim 1, wherein the gene related to innate immunity is TNF, IFN, p50, p65, IRF3, IL-6, IL-12, TLR, MyD88, or TRIF.

4. The agent according to claim 3, wherein the expression of a gene related to innate immunity is inhibited using siRNA.

5. The agent according to any one of claims 1 to 4, wherein the cells are human cells.

6. The agent according to claim 5, wherein the human cells are human somatic cells or human stem cells.

7. The agent according to claim 6, wherein the human somatic cells or human stem cells are human pluripotent stem cells, human pluripotent stem cell-derived differentiated cells, human mesenchymal stem cells, human myoblasts, human hematopoietic stem cells, human muscle cells, human blood cells, human fibroblasts, human nervous system cells, human epidermal cells, human epithelial cells, human endothelial cells, human osteocytes, human chondrocytes, or human adipocytes.

8. The agent according to claim 7, wherein the human somatic cells or human stem cells are human myoblasts, human mesenchymal stem cells, or human T cells.

9. The agent according to any one of claims 1 to 8, wherein the transgene is single-stranded or double-stranded.

10. The agent according to any one of claims 1 to 9, characterized in that the agent is administered with a transgene.

11. A method for producing transgenic cells, comprising administering the agent according to any one of claims 1 to 10.

12. A method for increasing the number of transgenic cells in vitro, the method comprises:
before or simultaneously with the introduction of genes into cells, administering
(i) an NFκB nuclear translocation inhibitor, or
(ii) an expression inhibitor of genes related to innate immunity.


SEQUENCE LISTING (14 sequences)

SEQ ID NO: 1 - RNA, 3420 nt, synthetic construct
  misc_feature 1..3420: note = GFP mRNA
  source 1..3420: mol_type = other RNA
atggtgagcaagggagaagagttatttactggtgtagttcctatattagtcgagctggacggggacgtgaacggacataaattttctgtgagcggggagggagaaggtgatgcgacttatggcaagttaacactaaagtttatttgcacaacaggcaaacttcctgtcccgtggcctactctggtaaccacgttaacttatggtgtgcagtgcttctctaggtatcccgatcatatgaagcaacacgatttcttcaagtcagctatgcccgagggttacgtgcaagagcgtactatattctttaaagatgatggaaactataaaacacgtgctgaagtcaaattcgaaggggacacgcttgtcaataggatcgaacttaaaggtattgacttcaaagaggacggcaacatcttaggacacaaactagaatacaactataactctcataatgtctatatcatggccgacaagcagaagaatggcatcaaagttaatttcaagataagacataacattgaagatgggagtgtccagttggccgaccattaccaacagaacacacccattggcgacggcccagttctgctgccagacaaccattacttaagtacccaaagcgccctctcaaaagatccaaacgaaaagcgcgatcacatggtattattggaatttgtgaccgcggcaggaatcaccttggggatggacgaactgtataagtgagcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcgttggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctactctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctccgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactaagcttatcgatg

SEQ ID NO: 2 - RNA, 3399 nt, synthetic construct
  misc_feature 1..3399: note = TERT mRNA
  source 1..3399: mol_type = other RNA
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 3 - DNA, 3399 bp, synthetic construct
  misc_feature 1..3399: note = hTERT gene
  source 1..3399: mol_type = other DNA
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 4 - DNA, 21 bp, synthetic construct
  misc_feature 1..21: note = TNF-alpha primer (forward)
  source 1..21: mol_type = other DNA
atcaatcggcccgactatctc

SEQ ID NO: 5 - DNA, 21 bp, synthetic construct
  misc_feature 1..21: note = TNF-alpha primer (reverse)
  source 1..21: mol_type = other DNA
gcaatgatcccaaagtagacc

SEQ ID NO: 6 - DNA, 20 bp, synthetic construct
  misc_feature 1..20: note = beta actin primer (forward)
  source 1..20: mol_type = other DNA
agaaaatctggcaccacacc

SEQ ID NO: 7 - DNA, 20 bp, synthetic construct
  misc_feature 1..20: note = beta actin primer (reverse)
  source 1..20: mol_type = other DNA
agaggcgtacagggatagca

SEQ ID NO: 8 - DNA, 20 bp, synthetic construct
  misc_feature 1..20: note = IFN-alpha primer (forward)
  source 1..20: mol_type = other DNA
ttctctgggctgtgatctgc

SEQ ID NO: 9 - DNA, 21 bp, synthetic construct
  misc_feature 1..21: note = IFN-alpha primer (reverse)
  source 1..21: mol_type = other DNA
ctgtccttcaggcaggagaaa

SEQ ID NO: 10 - DNA, 744 bp, synthetic construct
  misc_feature 1..744: note = CD19-scFV
  source 1..744: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgca

SEQ ID NO: 11 - DNA, 117 bp, synthetic construct
  misc_feature 1..117: note = CD28-hinge
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 12 - DNA, 81 bp, synthetic construct
  misc_feature 1..81: note = CD28-TM
  source 1..81: mol_type = other DNA
ttttgggtgctggtggtggttggtggagtcctggcttgctatagcttgctagtaacagtggcctttattattttctgggtg

SEQ ID NO: 13 - DNA, 123 bp, synthetic construct
  misc_feature 1..123: note = CD28
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 14 - DNA, 720 bp, synthetic construct
  misc_feature 1..720: note = EGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa